(19) United States
(12) Patent Application Publication
(10) Pub. No.: US 2004/0162596 A1
(43) Pub. Date: Aug. 19, 2004

(54) Methods and apparatus for performing photobiostimulation

(75) Inventors: Gregory B. Altshuler, Wilmington, MA (US); Ilya Yaroslavsky, Wilmington, MA (US); Michail Pankratov, Waltham, MA (US); Dov Gal, Brookline, MA (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: Palomar Medical Technologies, Inc., Burlington, MA

(21) Appl. No.: 10/680,705

(22) Filed: Oct. 7, 2003

Related U.S. Application Data
(60) Provisional application No. 60/416,664, filed on Oct. 7, 2002.

(51) Int. Cl. A61F 7/00; A61F 7/12

(52) U.S. Cl. 607/88; 607/96

(57) ABSTRACT

The present invention provides methods and devices for modulating the efficacy and/or increasing the efficiency of treatment of disease and/or cosmetic conditions through photobiostimulation combined with heating and/or cooling of the treatment region. In one aspect, methods and devices of the present invention are directed to modulating the efficacy of photobiostimulation in a target region by controlling the temperature in the region and/or its surrounding volume. According to some aspects of the present invention, tissue is heated such that biostimulation is applied to tissue that is hyperthermic. Alternatively, portions of the target region can be cooled to selectively target biostimulation to a specific region at a desired depth below the skin surface. A feedback mechanism is also provided so that the temperature of the target region can be selectively and accurately controlled.

### PRIORITY

The present invention claims priority to U.S. Provisional Application No. 60/416,664, filed Oct. 7, 2002 entitled “Methods and Apparatus for Performing Photobiostimulation.”

### BACKGROUND OF THE INVENTION

This invention is directed to methods and apparatus for performing photobiostimulation of tissue, and more particularly to methods and apparatus for performing temperature controlled photobiostimulation of tissue.

Low-power emitting lasers (i.e., typically less than 100 mW) have been used worldwide over the past three decades to treat a variety of clinical conditions. For example, light has been reported to stimulate DNA synthesis, activate enzyme-substrate complexes, transform prostaglandins and produce microcirculatory effects. There have been numerous reports of such effects resulting from irradiating endogenous chromophores (i.e., without application of exogenous photosensitizers) in cells or tissues.

The use of low-level light to achieve such photochemical responses is commonly referred to as photobiostimulation. In addition to laser light, photobiostimulation may be achieved using other monochromatic or quasi-monochromatic light sources (e.g., LEDs) or by suitably filtering broadband light sources (e.g., filtering fluorescent lamps, halogen lamps, incandescent lamps, discharge lamps, or natural sunlight). Biostimulation achieved by laser sources is also referred to as low-level laser therapy (LLLT).

Low-level light or low-level laser therapy stimulates the tissues and promotes healing by penetrating deep into the tissues initializing the process of photobiostimulation. The light energy is absorbed in cytochromes and porphyrins within cell mitochondria and cell membranes producing a small amount of singlet oxygen. Healing results from such treatments as demonstrated in many thousands of clinical study cases. Typically, patients can expect to feel noticeable improvement after four to six sessions for acute conditions and after six to eight treatments for chronic conditions. In many instances, photobiostimulation can be a viable alternative to surgery.

The photochemical process resulting from photobiostimulation is believed to involve the integration of photons into the cellular machinery of biochemical reactions. Generally, the principle of light absorption and integration of the photon energy into the cellular respiratory cycle is a well-known natural phenomenon. Photosynthesis and vision are two examples of this phenomenon. In these processes, the photoacceptor molecules are chlorophyll and rodopsin, respectively.

In the case of photobiostimulation, several concurrent mechanisms of action have been demonstrated in vitro. One example of such a mechanism involves cytochrome c oxidase, which is a primary cellular photoacceptors of low level light. Cytochrome c oxidase is a respiratory chain enzyme residing within the cellular mitochondria, and is the terminal enzyme in the respiratory chain of eukaryotic cells. In particular, cytochrome c oxidase mediates the transfer of electrons from cytochrome c to molecular oxygen. The involvement of cytochrome c is known to be central to the redox chemistry leading to generation of free energy that is then converted into an electrochemical potential across the inner membrane of the mitochondrion, and ultimately drives the production of adenosine triphosphate (ATP). Accordingly, it has been postulated that photobiostimulation has the potential of increasing the energy available for metabolic activity of cells.

It has been further demonstrated that photobiostimulation may be used to enhance cellular proliferation to achieve therapeutic effects. ATP molecules serve as a substrate to cyclic AMP (cAMP) which, in conjunction with calcium ions (Ca²⁺), stimulate the synthesis of DNA and RNA. cAMP is a pivotal secondary messenger affecting a multitude of physiological processes such as signal transduction, gene expression, blood coagulation and muscle contraction. Accordingly, it has been postulated that an increase in ATP production by photobiostimulation may provide a means to increase cell proliferation and protein production.

Light-stimulated ATP synthesis, such as that caused by photobiostimulation, is wavelength dependent. Karu (Lasers in Medicine and Dentistry. Ed. Z. Simunovic, Vitgraf:Rijeka, 2000, pp.97-125.) demonstrated in vitro that prokaryotic and eukaryotic cells are sensitive to two spectral ranges, one at 350-450 nm and another at 600-830 nm. Karu demonstrated that the light receptors of the red wavelengths are the semichinon type of the flavoproteins of the reductase (dehydrogenases) and the cytochrome a/a3 of cytochrome c. Cytochrome c oxidase in its oxidation form is the specific chromophore of 800 nm through 830 nm wavelength range.

Another mechanism of biostimulation involves causing a very limited irritation to the blood cells and walls in the vessels of the dermis. This results in a low-grade inflammatory/growth response. Inflammatory mediators are released through the vessel walls that stimulate fibroblast activity and eventually lead to a “healing” effect.

While the above mechanisms and positive effects have been demonstrated in numerous in vitro studies, results of clinical trials have been so far inconclusive. While some groups reported varying degree of success in the treatment of a range of conditions, others observed no or minimal effect. U.S. Pat. Nos. 5,514,168, 5,640,978, 5,989,245, 6,156,028, 6,214,035, 6,267,780, and 6,221,095, which are hereby incorporated by reference, provide examples of methods and devices for biostimulation. While various methods and devices of biostimulation exist in the art, more efficient and efficacious methods of treatment that yield quicker results with less treatment sessions are needed.

Photobiostimulation has been typically performed using relatively inexpensive sources, such as diode lasers or LEDs such as Ga—As and Ga—Al—As (e.g., emitting in the infrared spectrum (600-980 nm)). Existing sources of low power laser light and light emitting diodes (LEDs) deliver power levels ranging from 1 to 100 milliwatts; accordingly power densities necessary to perform photobiostimulative procedures are achieved by concentrating the light beam output into a very small spot sizes (typically less than 10 mm). This results in a typical power density at the skin surface in a range between 1 and 100 mW/cm². The small beam size makes a scanning device necessary to treat large areas. Treatment times used in most studies are in the range of 5 to 30 min and multiple treatments are often required.

There exists a need in the art for improved methods and devices for biostimulation that improve efficacy of treatment of disease and/or cosmetic conditions and, thus, will require less treatment sessions.

### BRIEF SUMMARY OF THE INVENTION

The present invention provides methods and devices for modulating the efficacy and/or increasing the efficiency of treatment of disease and/or cosmetic conditions through photobiostimulation combined with heating and/or cooling of the treatment region. In one aspect, methods and devices of the present invention are directed to modulating the efficacy of photobiostimulation in a target region by controlling the temperature in the region and/or its surrounding volume. According to some aspects of the present invention, tissue is heated such that biostimulation is applied to tissue that is hyperthermic. Alternatively, portions of the target region can be cooled to selectively target biostimulation to a specific region at a desired depth below the skin surface. A feedback mechanism is also provided so that the temperature of the target region can be selectively and accurately controlled.

The present invention is based in part on the discovery that heat enhances the effects of biostimulation. Heat enhanced biostimulation can take various forms. For example, heat may slow the repair of radiation-induced DNA damage, leaving more damage unrepaired and increased amounts of free radicals in the target region resulting in increased effects of biostimulation. Heat may also induce the production or activation of heat shock proteins or modify the rates of enzymatic processes. Currently, treatment sources and operating conditions used in conventional photobiostimulation provide negligible heating of treated tissue (e.g., less than 1° C. above normal body temperature).

In one aspect, the invention provides methods and devices for biostimulating a target region of a subject comprising irradiating a target region with a radiation, generated by a radiation source which has at least one selected wavelength component suitable for biostimulation, for a selected time duration and controlling a temperature of the irradiated target region with a source independent of said biostimulating radiation so as to modulate efficacy of said biostimulation. The time duration is chosen so as to cause biostimulation of the target region. In some embodiments, the target region is disposed at a depth below a skin surface of the subject. Time duration can be selected based on the desired application. Preferably time durations are chosen to be in a range of about 10 seconds to about one hour or in the range of about 10 minutes to about one hour. The temperature can be controlled, for example, by placing the target region in thermal contact with a surface having a selected temperature, by generating a flow of a fluid or air over the target region to be in thermal contact therewith, by applying electromagnetic or ultrasound radiation to the target region, or by applying a vaporizing cream, or a precooled and/or preheated cream or lotion to the target region. Those having ordinary skill in the art will appreciate that the other methods may also be utilized for controlling the temperature of the target region and/or its surrounding volume.

The wavelength component can be selected to be in a range of about 380 nm to about 1250 nm, in a range of about 380 nm to about 600 nm, in a range of about 380 nm to about 450 nm, in range of about 600 nm to about 700 nm, or in a range of about 760 nm to 880 nm depending on the desired application. The radiation source can preferably generate radiation with a narrow bandwidth, for example, a bandwidth less than about 100 nm.

The radiation can deliver a power flux in a range of about 1 to about 250 mW/cm² to the target region, or more preferably in a range of about 10 to about 100 mW/cm². The radiation can deliver an energy flux in a range of about 1 Joule/cm² to about 1000 Joules/cm², or more preferably in the range of about 1 Joule/cm² to about 100 Joules/cm², to the irradiated target region during irradiation time.

According to some aspects of the invention, the target region is irradiated by exposing it to a beam of radiation having a cross-sectional area in a range of about 1 cm² to about 10 cm². However, the beam's cross-section can be increased based on the application.

In some aspects, the step of controlling temperature includes heating the irradiated target region, referred to as hyperthermia herein, so as to increase efficacy of the biostimulation. The heating step can be performed by contact heating, convection, or application of electromagnetic radiation, such as ultrasound, microwave, or infrared energy. Hyperthermia is defined herein to be a temperature greater than normal body temperature. Normal body temperature can range from 36.1° C. to 37.2° C. depending on the time of day. Accordingly, the temperature of the surface area of the target region to which biostimulation is applied in practice of the invention can be increased to 37-50° C. and preferably 37-45° C. In some embodiments, the temperature of the target area can be increased to be within a range of about 37-42° C. or, alternatively, be within a range of about 38-42° C. In other embodiments, the temperature of the target area is increased to be within a range of about 38-41° C. The temperature is preferably elevated above normal body temperature, but below a temperature at which pain and denaturation of a significant concentration of critical biomolecules occurs.

Further aspects of the present invention are directed to cooling a target region to which biostimulative radiation is applied. According to at least some aspects of the invention, a portion of the region of tissue is cooled such that the skin is protected from heat damage and/or the efficacy of biostimulation in the region is reduced to control depth of treatment. The target region can be cooled to a value in a range of about 0° C. to about 36° C. , or about 10-36° C., or about 15-36° C., or about 20-36° C., or about 28-36° C.

In some embodiments, controlling the temperature comprises utilizing a separate radiation source to heat the target region irradiated with biostimulating radiation. The separate radiation source can include a narrowband source or broadband source. The separate radiation source can generate radiation having one or more wavelength components in a range of about 380 nm to about 2700 nm, preferably in a range of about 1000 nm to about 1250 nm, or more preferably in a range of about 700 nm to about 900 nm.

In one aspect of the invention, the step of controlling the temperature of the irradiated target region comprises heating a first selected portion of the target region and cooling a second selected portion of the target region. Heating and cooling can be either simultaneous or sequential. Beneficial effects may result from rapidly changing or fluctuating the temperature of the target region before, during, or between irradiation sessions.

In another aspect of the invention, a method of biostimulating a target region of a patient disposed at a depth below the patient's skin is disclosed. The method includes exposing a portion of the patient's skin for a selected time duration to a radiation having at least one selected wavelength component capable of penetrating to a depth associated with the target region so as to irradiate the target region. The temperature of a volume of the patient through at least a portion of which the radiation traverses to reach the target region is controlled so as to modulate biostimulation within that volume relative to the target region. The wavelength component and the time duration are chosen to cause biostimulation within the target region. The temperature can be controlled to cool the volume and decrease biostimulation therein. For example, the temperature of the volume can be decreased to be within the range of about 0° C. to about 36° C. or preferably in a range of about 15° C. to about 36° C. The wavelength component can be selected to be in a range of about 380 nm to about 1250 nm or more specific ranges described herein. The radiation source can generate radiation with a narrow bandwidth that can be less than about 100 nm.

In yet another aspect, the invention discloses a device for biostimulating a patient's target region that includes a first source for generating electromagnetic radiation having one or more wavelength components suitable for causing biostimulation in the target region; a radiation guidance device optically coupled to the source for delivering the radiation to the target region; and a second source in communication with the target region for controlling a temperature of the target region in order to modulate efficacy of biostimulation caused by the electromagnetic radiation. The first source can generate radiation having a narrow bandwidth, for example, less than about 100 nm. The first source can generate radiation having one or more wavelength components in a range of about 380 nm to about 1250 nm. The second source can include a source of electromagnetic radiation generating radiation suitable for heating the target region so as to enhance the efficacy of biostimulation. For example, the second source can generate one or more wavelength components in a range of about 380 nm to about 2700 nm.

In a related aspect, the device can further include an optical fiber coupled at an input thereof to the first radiation source and an output thereof to the radiation guidance device, for example, a lens system, so as to direct light generated by the radiation source to the lens system. The lens system can have at least one movable lens to allow adjusting a cross-sectional area of a radiation beam generated by the first source for irradiating the target region. The lens system can comprise a Fresnel lens.

In another aspect, the radiation guidance device may include a beam splitter adapted to receive a radiation beam from the first source in order to generate a plurality of beam portions, and one or more reflective surfaces optically coupled to the beam splitter to direct one or more of the beam portions to a surface of the patient's skin so as to irradiate the target region. The reflective surfaces can allow a substantially uniform illumination of the skin surface. The beam splitter can be, for example, a prism, and at least one of the reflective surfaces can exhibit a curved profile.

In another aspect, the invention provides a method of biostimulating a subject's target region that includes irradiating the target region with radiation having one or more wavelength components suitable for causing biostimulation within the target region, and actively controlling a temperature of at least a portion of the target region to ensure it remains within a pre-defined range of an operating temperature in order to modulate efficacy of biostimulation within the target region. The step of actively controlling the temperature can include measuring a temperature of a portion of the patient's skin in thermal contact with the target region and comparing the measured temperature with at least one pre-defined threshold. The amount of heat delivered to or extracted from the target region can be controlled in response to the comparison of the measured temperature with the pre-defined threshold.

In yet another aspect, the invention provides a method for biostimulating a plurality of target regions of a subject by moving a radiation source over a portion of the subject's skin so as to irradiate sequentially a plurality of target regions with radiation having at least one wavelength component suitable for causing biostimulation. The moving of radiation source can be performed at a rate selected to expose each of the regions to sufficient radiation for causing biostimulation therein. The temperature of the target regions can be controlled by a source independent of the biostimulating radiation so as to modulate efficacy of biostimulation within each of the target regions. The moving radiation source can expose each target region, once, or alternatively, multiple times, to biostimulative radiation.

### BRIEF DESCRIPTION OF THE DRAWINGS

FIG. 1 schematically illustrates an embodiment of the invention in which a target region, which extends from the surface of the skin to a selected depth, is heated such that biostimulation is applied to a hyperthermic volume of tissue;

FIG. 2 schematically illustrates another embodiment of the invention in which biostimulation is applied to a heated target region in proximity of the skin surface while biostimulation is applied simultaneously to an unheated volume below the target region;

FIG. 3 schematically illustrates another embodiment of the invention in which photobiostimulation is generated in a volume of tissue at a depth region below the surface of skin while cooling is applied to the surface of skin;

FIG. 4 schematically illustrates another embodiment of the invention in which biostimulation is applied to a hyperthermic volume of tissue that is at a selected depth below the surface of the skin, and unheated volumes are located above and below the hyperthermic volume of tissue;

FIG. 5 schematically illustrates another embodiment of the invention in which enhanced biostimulation occurs in a first volume of tissue, which is both hyperthermic and located at a selected depth below the surface of the skin, and biostimulation (without hyperthermia) also occurs in a second volume of tissue that is located below the first volume of tissue;

FIG. 6 is a graph of selected temperature profiles of type II skin using exemplary wavelengths of monochromatic light without skin cooling;

FIG. 7 is a graph of selected temperature profiles of type II skin using exemplary wavelengths of monochromatic light with parallel skin cooling;

FIG. 8 is a schematic diagram of a light projection system for biostimulating a target region, according to the teachings of the invention;

FIG. 9A is an exemplary embodiment of a light projection system for forming substantially uniform illumination of a non-flat surface;

FIG. 9B is a schematic diagram of an exemplary beam splitter suitable for use in a device according to the teachings of the invention;

FIG. 10 is a schematic diagram of another exemplary embodiment of a light projection system for forming substantially uniform illumination over a non-flat surface;

FIG. 11A is a schematic diagram of another embodiment of a light projection system according to the teachings of the invention that utilizes a rotatable head to provide substantially uniform illumination to a non-flat surface, where the rotatable head is positioned to direct light onto a front portion of the non-flat surface

FIG. 11B is a schematic diagram of another embodiment of a light projection system according to the teachings of the invention that utilizes a rotatable head to provide substantially uniform illumination to a non-flat surface, where the rotatable head is positioned such that light is directed onto a first side portion of non-flat surface;

FIG. 11C is a schematic diagram of another embodiment of a light projection system according to the teachings of the invention that utilizes a rotatable head to provide substantially uniform illumination to a non-flat surface, where the rotatable head is positioned such that light is directed onto a second side portion of non-flat surface;

FIG. 12A is a graph of the temperature of type II skin surface as a function of time of exposure to a 800 nm radiation at a flux of 680 mW/cm², wherein the beam has a diameter larger than 2.5 cm;

FIG. 12B is a graph of temperature profiles in which the type II skin surface is cooled and kept at 36° C. while being exposed to different wavelengths of radiation according the invention;

FIG. 13A is an exemplary embodiment of a light projection system for use in the invention;

FIG. 13B depicts an exemplary set of lens parameters according to the invention;

FIG. 14 illustrates an exemplary embodiment of a device, according to the invention, capable of irradiating a target region and controlling the temperature of that region through a feedback mechanism; and

FIG. 15 illustrates an exemplary embodiment of a device, according to the invention, capable of irradiating a target region using a 2D matrix of radiation sources.

### DESCRIPTION OF THE INVENTION

In one aspect, the present invention is directed to controlling the efficacy of photobiostimulation in a target region by controlling the temperature of that region. The heating or cooling of the target region, i.e., patient's skin, hair, eye, teeth, nails, or other body tissue, can trigger biological processes within the body that can work synergistically with photobiostimulation to yield better, more efficient results. The temperature of the target region is modulated during, prior to, or between photobiostimulation. The synergy between irradiation and temperature modulation can vary depending on the order of application and/or the disease or cosmetic condition to be treated. In a preferred embodiment, modulation of the temperature and irradiation occurs simultaneously.

In one embodiment, the temperature of the target region is increased. Heating of tissue, hyperthermia, leads to increased local tissue perfusion and increased blood and lymph circulation. The increase in blood flow has multiple effects on photobiostimulated tissues. The cellular biochemical reactions of biostimulation are accelerated since the rates of some enzymatic reactions increase at higher temperatures. Additionally, more oxygen is available for the increased cellular metabolism, and the toxic by-products of metabolism are removed more readily, through the blood and lymphatic circulation. In addition, heating of blood vessels can increase vessel wall and/or cell wall permeability, which may result in improved delivery of therapeutic additives (i.e., vitamins, antioxidants, lotions, etc.) or drugs to the target area. For example, topical drugs may be enclosed in thermosensitive liposomes that selectively release their drug content when exposed to heat.

Hyperthermia in a tissue to be treated may be achieved by use of any suitable technique, including but not limited to use of contact heating, convection (i.e., by heated air), or application of electromagnetic radiation. In some embodiments, hyperthermia in a tissue to be treated is achieved by absorption of a portion of the incident electromagnetic radiation from a biostimulative source used to biostimulate the tissue. For example, absorption of electromagnetic radiation may be by tissue chromophores such as melanin, hemoglobin, water, lipids or other chromophores which cause a photothermal interaction leading to an increase in tissue temperature. Hyperthermia generates a cascade of events, such as increasing vasodilation, increasing blood circulation, increasing production of heat shock proteins, which can act synergistically with photobiostimulation resulting in improved efficacy of treatment.

Additionally, local hyperthermia is known to activate the heat shock (HS) response, thermotolerance and hormesis (P. Verbeke, et al. Cell Biol Inter. 2001; 25:845-857). The phenomenon of thermotolerance is defined as the capacity of cells, following a cycle of heat stress and recovery, to survive a second stress, which would otherwise be lethal. Mild heat shock treatment may prevent cell death from a variety of subsequent stresses. Similar to exposure of cells and organisms to stresses such as caloric restriction, exercise, oxidative and osmotic stress, heavy metals, proteosome inhibitors, amino acid analogues, ethanol, and metabolic poisons, heat shock treatment induces a cellular stress response leading to the preferential transcription and translation of heat shock proteins (HSPs). Numerous families of HSPs have been identified (P. Verbeke, et al. Cell Biol Inter. 2001; 25:845-857).

When a cell encounters a stressor, modifications of the cytoskeleton, cytoplasmic structures, cell surface morphology, cellular redox status, DNA synthesis, changes in protein metabolism and protein stability occur. Such stress generates a molecular remodeling or damage, especially abnormal folded proteins, which can aggregate and initiate a sequence of stress responses. The induction of the HS response occurs through molecular links between the environmental stresses and the stress response. When stress alters protein folding, or proteins begin to unfold and denature, HSPs have been shown to assist in protein refolding, to protect cellular systems against protein damage, to solubilize aggregates to some extent, to sequester overloaded and damaged proteins into large aggregates, to target fatally damaged proteins for degradation, and to interfere with the apoptotic progression (P. Verbeke, et al. Cell Biol Inter. 2001; 25:845-857).

HSPs that are involved in the renaturation of unfolded proteins are referred to as chaperones. Chaperones recognize and bind to other proteins when they are in non-native conformations and are exposing hydrophobic sequences. Such HSPs protect many different systems involved in maintenance of cellular functions. Some HSPs induce an increase in the cellular glutathione (GSH) level leading to the protection of the mitochondrial membrane potential during stress. Members of the HSP70 and HSP90 families are associated with the centrosome. They are known to bind and stabilize actin, tubulin and the microtubules/microfilament network, playing a role in the cellular morphology and transduction pathways.

Thermotolerance is believed to be mainly due to the orchestrated regulation of expression and accumulation of various HSPs in the endoplasmic reticulum and in the cytosol, leading to marcromolecular repair mechanisms as a defensive strategy against subsequent challenges. A further characteristic of responses to HS is that various HSPs are soluble and transfer across the cell membrane to other adjacent cells. Consequently, the protective stress response is transferable to neighboring cells that might not be able to mount such a reaction. Accordingly, a next treatment can be done with higher temperature. This mechanism can be used to increase the maximum tolerable incident power applied to the skin surface. Specifically, the power can be increased gradually, allowing the organism to adapt to the thermal stress and thus survive a higher level of hyperthermia than would be possible without such adaptation.

In addition to the HSP-dependent effects described above, HSP-independent effects may arise from hyperthermia. Other mechanisms of stress tolerance include the synthesis of osmotic stress protectants, modifications of the saturation of cell membrane lipids, and expression of enzymes such as radical scavengers.

Similar to thermotolerance, hormesis is a response to repeated mild stress, which enhances cellular defense processes. Hormesis is a process by which cells adapt to gradual changes in their environment so as to be able to survive subsequent exposure to otherwise lethal conditions. Such a phenomenon has been observed in relation to irradiation, toxins, heat shock and other stresses. Ratan et al observed anti-aging hormetic effects of repeated mild HS on human fibroblasts (Rattan et al. Biochem Mol Biol Int 1998;45:753-759). Kevelaitis et al showed that local and brief application of heat (42.5° C. for 15 minutes) to the myocardium improved cardiac systolic and diastolic functions (Kevelatis et al. Ann Torac Surg 2001;72:107-113).

The above indicates that systems according to aspects of the present invention should improve the clinical utility and outcome of biostimulation therapy. It further appears that aspects of the present invention provide synergistic effects of photochemical biostimulation of cells and mild tissue hyperthermia, which stimulate HSP-dependent and HSP-independent thermotolerance, and/or hormesis. This synergism may lead to repair of cell damage and improved functionality of compromised cells. Those effects may help in the treatment of conditions associated with infection, acute and chronic inflammation, micro circulatory stagnation, and may also stimulate regeneration and rejuvenation of tissues subjected to degenerative processes, for example, by stimulating fibroblast proliferation, or by increases in growth factors eventually leading to new synthesis of intracellular and extracellular proteins, glycoproteins and lipid soluble molecules. Additional aspects of the present invention control the effectiveness of biostimulation provided by selectively delivered photobiostimulative light to deep structures through the use of temperature control (e.g., via heating and/or cooling of a tissue surface) and/or through control of radiation spot size.

In another aspect of the invention, a means for controlling specific mechanisms of photobiostimulation in order to achieve a desired therapeutic effect is provided. It is known that the biological response to photobiostimulation can vary as a function of the state of the biological system. For example, human fibroblasts can display a diversity of responses when exposed to outside stimuli (Lasers in Medicine and Dentistry. Ed. Z. Simunovic, Vitgraf:Rijeka, 2000, pp.97-125). In particular, both stimulation of proliferation of fibroblasts and an increase in production of type I collagen have been reported. However, production of collagen was affected in a manner inverse to the effect on cell proliferation, i.e., when proliferation increased, production of collagen decreased. Therefore, one can manipulate the state of the target system in order to channel the action of biostimulation into a desired pathway. One factor greatly influencing the state of the biological system is the temperature. The present invention provides a way to fine-tune the resulting biological response through the control of the temperature of the biostimulated area.

The present invention provides methods and devices for modulating the efficacy of biostimulation. The term “modulates efficacy” as used herein refers to a change of the resulting biostimulation effects of greater than 10%, preferably greater than 20%, more preferably greater than 30%, more preferably greater than 40%, more preferably greater than 50%, more preferably greater than 60%, more preferably greater than 70%, more preferably greater than 80%, more preferably greater than 90% and most preferably greater than 100%. The efficacy of biostimulation can be measured in terms of the time necessary to achieve a desired outward appearance, i.e., removal of wrinkles or scar tissue, or a time needed for patient satisfaction, i.e., pain relief, or the rate of the underlying enzymatic mechanisms. For example, substantially increasing the efficacy of biostimulation of a target region can refer to an increase in the rate of enzymatic processes in that target region of more than 10% relative to unstimulated steady-state condition. The rate of the enzymatic processes can be determined using any of the methods known in the art (See, for example, T. Bugg, An Introduction to Enzyme and Coenzyme Chemistry, Blackwell, 1997; Wright et al. Photochem Photobiol. 2002 July;76(1):35-46; Koekemoer et al. Comp Biochem Physiol B Biochem Mol Biol. 2001 July;129(4):797-807). For example, the enzymatic activity of cytochrome c oxidase or the rate of radical production, i.e., singlet oxygen, can be used as a measure of biostimulation in the target region. Free radical production can be determined by measuring superoxide dismutase (SOD) and catalase or glutathione peroxidase levels in the cytoplasm. In addition, indirect measures of free radical production can be used such as through consumption of antioxidants.

The mechanisms described above are illustrative, and are not exhaustive. Accordingly, they should not be considered as limiting the scope of the presented invention. Additionally, because photobiostimulation is an emerging field, the theories regarding the mechanisms achieving a given result are in many instance speculative.

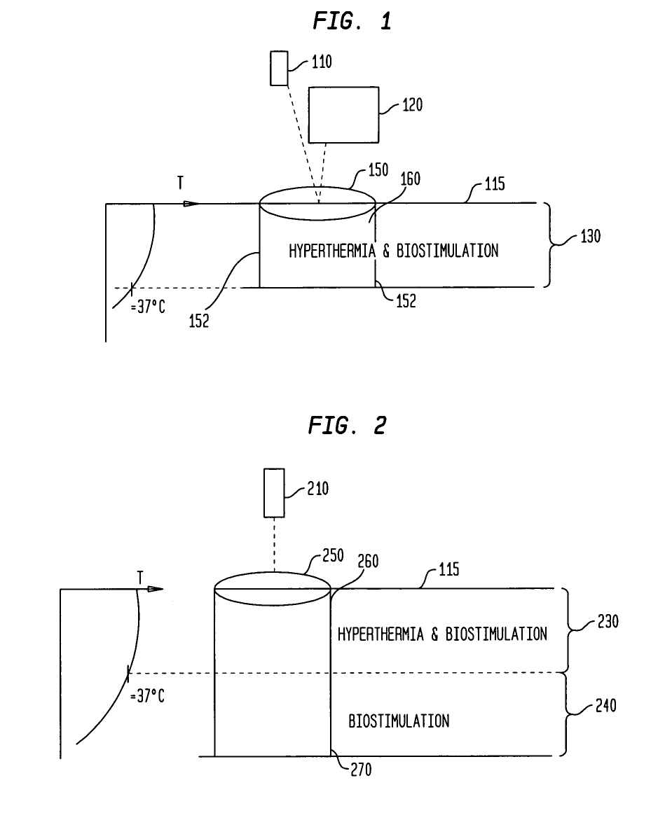

FIGS. 1-5 are schematic cross-sectional views of systems that illustrate five exemplary treatment scenarios for achieving photobiostimulation and temperature control (e.g., hyperthermia and/or hypothermia) of a volume of tissue according to at least some aspects of the present invention.

In each of the treatment scenarios, biostimulation is achieved by applying electromagnetic radiation to the skin surface from a source suitable for achieving biostimulation. For example, a suitable source may comprise a narrow bandwidth source, such as a monochromatic or quasi-monochromatic source. Appropriate sources can include lasers, LEDs or suitably filtered broadband sources (e.g., filtered lamps). The invention can also utilize a 2D matrix of radiation sources. A suitable narrow bandwidth source preferably has a bandwidth (i.e., wavelength range) of less than approximately 100 nm, preferably below approximately 20 nm and more preferably below approximately 10 nm. The wavelength may be selected to achieve any known biostimulative effect. The wavelength of the radiation may be, for example, in a range of 380-2700 nm. For example, radiation with a wavelength in a range of about 380-600 nm can be utilized for treating superficial tissues, while radiation with a wavelength in a range of about 600-1250 nm can be utilized for deep tissues. In an exemplary embodiment, preferred wavelength ranges that can be utilized for biostimulation are 380-450 nm, 600-700 nm, and 760-880 nm. However, the choice of wavelength depends on the specific application. Biostimulation has uses in cosmetics, dentistry, dermatology, ENT (ear, nose, and throat), gynecology, and surgery.

With reference to FIG. 15, in one exemplary embodiment, a 2D matrix of radiation sources can be employed to irradiate a target region to cause biostimulation therein while simultaneously, or in separate time intervals, delivering heat thereto. The exemplary radiation matrix 1500 includes a plurality of radiation sources 1510 (depicted as larger circles) that provide radiation with one or more wavelength components suitable for causing biostimulation in tissue, and a plurality of separate radiation sources 1520 (depicted as smaller circles) that can generate radiation with spectra suitable for heating a target region. A variety of radiation sources, such as LED or lasers, can be utilized for forming the 2D radiation matrix 1500.

Examples of applications of aspects of the invention include, but are not limited to, skin texture improvement, scar removal or healing, wrinkle removal, skin tightening, skin elasticity improvement, skin thickening, skin rejuvenation, cellulite treatment/fat reduction, vascular and lymph regeneration, subcutaneous collagen structure improvement, acne treatment, psoriasis treatment, fat reduction, hair growth stimulation, treatment of alopecia, treatment of lentigo senile, treatment of striae, pain relief, wound healing, healing of epidermis and dermatitis, treatment of eczema, treatment of decubitus ulcer, healing of haematoma, treatment after skin resurfacing, odor reduction, muscles contraction relaxation, reduction of gum inflammation, reduction of pulpitis, treatment of herpes, treatment of alveolities, aphtae and hyperemia, reduction of oedema, drum healing, treatment of tinnitus, reduction of microscars and polyposis, treatment of adnexitis, bartholinitis, cervicitis, epiziotomy, HPV, menorrhagia, and parametritis, and vulvitus. Non-limiting wavelength ranges that can be used to treat a variety of diseases and cosmetic conditions can be found in Table 1.

TABLE 1
Examples of wavelength ranges useful for the treatment
of specific diseases and cosmetic conditions.
Dermatology/Cosmetology

| | |
|---|---|
| Acne | 390-450 and 600-700 nm |
| Scars | 380-420, 620-680 and 760-830 nm |
| | (depending on scar nature) |
| Wrinkles | 620-680 and 760-880 nm |
| Cellulite | 760-880 nm |
| Striae | 760-880 nm |
| Lentigo senile | 600-700 nm |
| Alopecia | 620-680 and 760-880 nm |
| Skin rejuvenation | 600-700 and 760-880 nm |
| Hair growth stimulation | 600-700 and 760-880 nm |
| Psoriasis | 600-700 nm |

Dentistry

| | |
|---|---|
| Gingivitis | 380-450 and 600-700 nm |
| Gum inflammation | 380-450 and 600-700 nm |

Other

| | |
|---|---|
| Burns | 760-880 nm |
| Pain relief | 760-880 nm |
| Wound healing | 380-1250 nm (depending on wound nature) |

The treatment time is generally selected based on the time necessary to achieve hyperthermia of the tissue to be treated and the time necessary to irradiate the volume of hyperthermic skin with biostimulative radiation for a time sufficient to achieve a desired photobiochemical output.

According to some aspects of the invention, the time necessary to irradiate a volume of hyperthermic skin with biostimulative radiation can be determined using an assumption that there are approximately 10²³ molecules/cm³ in human tissue, and that a minimum of one photon is to be delivered to each molecule during the course of a single photobiostimulative treatment. For example, for a 1 cm³ treatment volume, 10²³ photons must be delivered. Assuming uniform distribution of the absorbed photons and that light is delivered through a 1 cm² window, the light fluence at the skin surface is equal to 10²³ times the energy in one photon of the monochromatic light, and the fluence divided by the light power output of the source determines the typical minimum treatment time. Typical treatment times are 10 seconds to 60 minutes. In some embodiments, the pulse duration is between 1 min to 1 hour. In other embodiments, the pulse duration is between 10 min to 1 hour. Treatments can be performed as often as necessary. For example, treatment may occur 5 to 10 times, with 1 day interval between treatments. The typical amount of total energy delivered to the target area can range from 1 J/cm² to 1 KJ/cm² and preferably is between about 1 J/cm² to 100 J/cm².

According to the present invention, hyperthermia can be achieved by any known means of achieving hyperthermia at the depth indicated in each of the scenarios. In the case of photohyperthermia, the source may be a broadband radiation source or a narrowband radiation source, and may be pulsed or continuous wave (cw). In some embodiments, pulsed light may be synchronized to a biological period of a patient (e.g., the patient's heart pulse, biological cycle). Further details regarding photohyperthermia are discussed below.

Exemplary ranges for parameters (e.g., wavelengths fluxes, temperatures, areas) described herein below for achieving temperature control and biostimulation indicate values which may be used to achieve a specified treatment; the values to be utilized for a specific treatment will depend on many factors including, but not limited to, the patient's skin type, the part of the patient's body being treated, the desired treatment, the depth of the treatment, the temperature of the treatment volume, etc. Additionally, it is to be appreciated that parameters are also interrelated. For example, energy/fluence and time of application are inversely related, one increasing as the other decreases in order to provide a desired number of photons at a target volume. Examples of parameters which provide desired results are provided herein and parameters for other treatments can be determined by one of ordinary skill in the art from the information provided herein and/or empirically.

FIG. 1 illustrates an exemplary embodiment of the invention in which a volume of tissue 160 is heated such that biostimulation is applied to a hyperthermic volume of tissue, wherein volume of tissue 160 extends from the surface of skin 115. Volume of tissue 160 is defined by a depth region 130 and a skin surface area 150. While the side 152 of volume of tissue 160 is illustrated as perpendicular to the surface of skin 115, it is to be understood that the area of treatment in FIG. 1, as well as those described below with reference to FIGS. 2-5, will typically increase with depth below the skin surface due to scattering of light by tissue. Additionally, while the boundaries of the volume of tissue 160 are illustrated with continuous lines, it is to be understood that the actual volume of treatment may be highly irregular, and regions of tissue outside of such bounds may receive both biostimulation and hyperthermia; however, biostimulation and/or hyperthermia may be to a lesser degree than for tissue in volume of tissue 160.

Biostimulation may be achieved using radiation from a suitable photobiostimulative source 110 as described above. For example, source 110 delivers radiation to the skin surface 115 with a flux in the range of about 1-250 mW/cm², and preferably in the range of about 10-100 mW/cm². Depth region 130 over which biostimulation is achieved is determined by the flux, the wavelength of light from source 110, and the size of area 150. For example, irradiation with radiation having a wavelength of 380-1250 nm at a flux 1-250 mW/cm² will achieve biostimulation to a depth up to 10 mm for a beam having a diameter of greater than 1 cm. While area 150 is illustrated as circular, it is to be understood that area 150 (and the other skin surface areas described below with reference to FIGS. 2-5) may be oval, square, rectangular, hexagonal or have any other suitable shape. Source 110 may be operated in contact with surface of skin 115 or project radiation onto surface of skin 115 from a distance.

Hyperthermia, an increased temperature, in volume of skin 160 may be achieved by any known source 120 capable of raising the temperature of volume 160 to a value within a range of about 37-50° C. and preferably about 37-45° C. Normal body temperature can range from 36.1° C. to 37.2° C. depending on the time of day. In some embodiments, the temperature of the target area can be increased to be within a range of about 37-42° C. In some embodiments, the temperature of the target area is increased to be within a range of about 38-42° C. In other embodiments, the temperature of the target region is increased to be within the range of about 38-41° C. In other embodiments, the temperature of the target region can be increased to about 38° C. In yet other embodiments, the temperature of the target region can be increased to about 39° C. In yet another embodiment, the temperature of the target region can be increased to about 40° C. For example, hyperthermia may be achieved by projecting hot air onto area 150, applying AC or DC electrical current, or using a conductive heat source (i.e., a device, such as a heated plate or heating pad, in contact with surface 115). Further examples of heating a tissue include using ultrasound and microwave radiation, as described in U.S. Pat. Nos. 5,230,334, and 4,776,086, respectively, herein incorporated by reference. If contact heating is desired, the heating source may be transparent to the biostimulative radiation such that the biostimulation can be provided to tissue through the heating source. Heating can be applied before, during or between photobiostimulation treatment sessions.

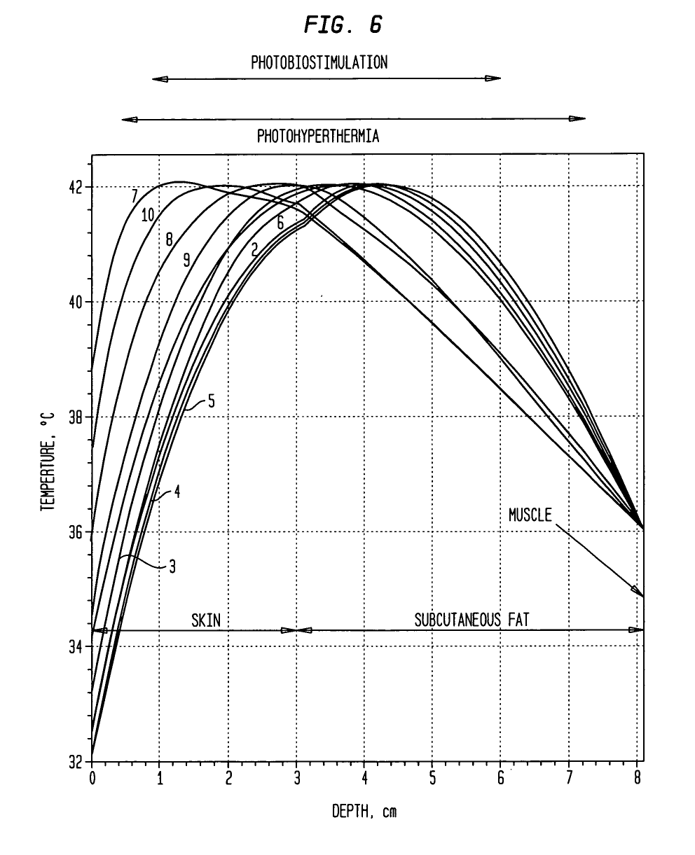

Optionally, source 120 may be a radiative source capable of achieving hyperthermia. Hyperthermia achieved using radiation is also referred to as photohyperthermia. A radiative source 120 may be any suitable radiative source that does not interfere with achieving biostimulation. To achieve hyperthermia, heating can be obtained using a broadband source or a narrowband source selected to achieve a desired temperature of tissue. Hyperthermia may be achieved using any suitable wavelength or wavelengths of electromagnetic radiation; for example, the radiation may be in the wavelength range 380-2700 nm; or preferably in the range 500-1250 nm, and more preferably in the ranges 650-900 nm and/or 1000-1250 nm. For example, the sources included in FIG. 6 may be combined in a weighted manner to provide a suitable temperature profile. A radiative source 120 may be operated in contact with surface of skin 115 or project radiation onto surface of skin 115 from a distance.

It is believed that a radiative source 120 will not interfere with achieving biostimulation if the spectral density of the combined output of biostimulative source 110 and source 120 is predominated by wavelengths that effect biostimulation. For example, the spectral density of the wavelengths in the band that effects biostimulation is 100 times greater than the spectral density of light in any other band, and preferably greater than 1,000 times. The phrase “spectral density” is herein defined to refer to the number photons in a specified bandwidth (e.g., the bandwidth at which biostimulation is achieved).

Biostimulation according to aspects of the invention may be achieved using sources applied in a conventional small area of irradiation (e.g., a round area having a spot size less than 10 mm² in diameter), or a larger area (e.g., a round area having a spot size 1 cm²-200 cm² or more up to and including the entire human body). Similarly, photohyperthermia according to aspects of the invention may be achieved using sources applied using a conventional small area (e.g., a round area having a spot size less than 10 mm in diameter), or a larger area (e.g., a round area having a spot size 1 cm²-200 cm² or more). Large areas offer advantages, including but not limited to, reduced treatment time. For example, large areas may be used to treat large areas of tissue such as a face, neck, back or thigh. Methods of achieving a large area of irradiation are described in greater detail with reference to FIGS. 8-11 and 13 below.

The present invention recognizes that boundary effects diminish as the volume to be irradiated increases. As the volume of the target region increases, the probability that the scattered radiation will remain within the irradiated volume also increases. Therefore, radiation can penetrate the target tissue to a greater depth when a larger beam of irradiation and/or a larger target area is used. Accordingly, in some embodiments, where treatment is to be affected to a significant depth in the tissue, a large area of illumination is used to effect the treatment. In contrast, conventional biostimulation apparatuses have used narrow incident beams, which are strongly attenuated such that the photons comprising the beam do not reach deeply into the dermis and subcutaneous tissue (and/or into muscles and bones) in high enough concentration to achieve the desired biostimulation. Additionally, in a conventional biostimulation apparatus, since only small areas are treated at a given time, the beneficial effect arising from the treatment of large areas of tissue are nonexistent. In some embodiments, photobiostimulative radiation is directed onto the skin surface using an area of illumination greater than approximately 0.8 cm² (e.g., a circular spot size greater than 1 cm²) and preferably greater than 1.6 cm² to provide biostimulation to tissue at relatively large depths below the skin surface, and to achieve time efficiencies resulting from treating a large area at one time. In one aspect, the present invention provides devices capable of providing such treatment.

FIG. 2 illustrates another embodiment of the invention in which a volume of tissue 260 is heated such that biostimulation is applied to a hyperthermic volume of tissue 260, wherein volume of tissue 260 is adjacent to the surface of skin 115, and a volume of tissue 270 receiving biostimulation (without hyperthermia) is located below volume 260. Volume of tissue 260 is defined by a depth region 230 and an area 250. According to this aspect of the invention, the same light source 210 is used to produce both hyperthermia and biostimulation of volume of tissue 260. Light source 210 also produces biostimulation in volume 270 in a depth 240.

An additional advantage of embodiments according to this aspect of the invention is that the depth of the biostimulation zone is effectively increased by increasing the flux of source 210 relative to the flux provided in FIG. 1. For example, an increase of flux incident on skin surface 115 from 100 mW/cm² to 200 mW/cm² is sufficient to induce pronounced hyperthermia, and will also increase effective biostimulation depth by up to 30% (i.e., an increase of the total biostimulation depth including depth regions 230 and 240 when compared to depth region 130 in FIG. 1).

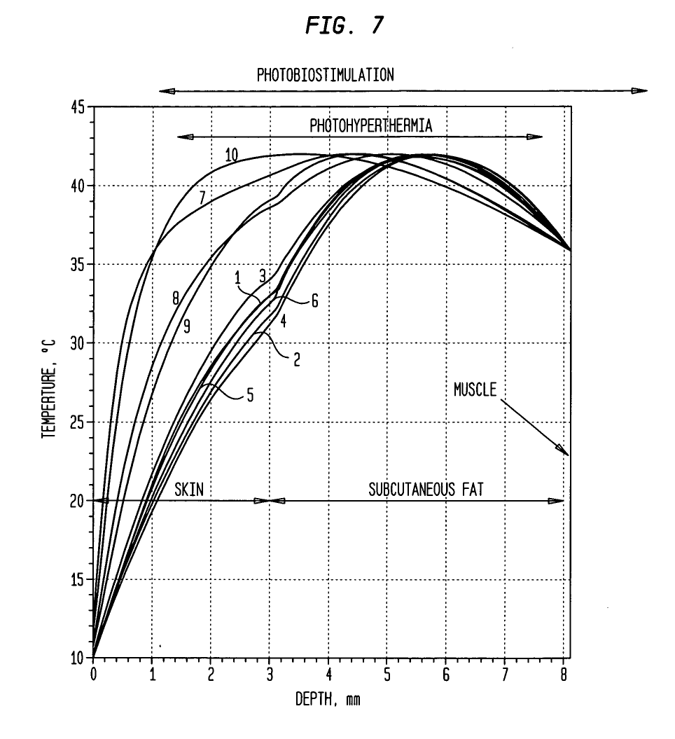

Hyperthermia and biostimulation are achieved in volume of tissue 260 by directing electromagnetic radiation from a narrowband source 210 onto an area 250. The wavelength of source 210 is selected to achieve a desired photobiostimulative result, and flux of source 210 is chosen to achieve a selected temperature profile as indicated by FIGS. 6 and 7. Biostimulation in volume 270 (defined by depth region 240 and area 250) is achieved where the intensity of light is sufficient to achieve biostimulation, but not sufficient to achieve a hyperthermic temperature (i.e., the temperature is less than 38° C.) as indicated in FIG. 2. It is to be appreciated that the effect of biostimulation is weaker in depth region 230 than in depth region 240 due to the absence of hyperthermia in depth region 240.

Biostimulation and photohyperthermia according to the second aspect of the invention, may be achieved using a conventional small area of irradiation (e.g., a round area having a spot size less than 10 mm in diameter), or a larger area (e.g., a round area having a spot size larger than 1 cm², up 200 cm² or more). Generally, the larger the area, the deeper depth regions 230 and 240 extend below surface 115 due to a reduction in the effect of scattering. For example, irradiation with a wavelength of 600-1250 nm at a flux 0.1-1.0 W/cm², and a spot size 1-200 cm after 80 seconds of exposure will achieve heating and biostimulation to a depth up to 30 mm and biostimulation (without hyperthermia) from 30 mm-50 mm.

FIGS. 6 and 7 present graphical data for achieving a selected temperature profile using exemplary wavelengths of monochromatic light without skin cooling (FIG. 6) and with parallel skin cooling (FIG. 7). Specifically, the numbered entries in Tables 2 and 3 describe the flux at the skin surface and the time necessary to achieve a correspondingly-numbered steady-state temperature profile in FIGS. 6 and 7, respectively. It is to be understood that the wavelengths in FIGS. 6 and 7 are exemplary and light of any suitable wavelength may be used to achieve hyperthermia. Exemplary profile 7, in FIG. 6, illustrates hyperthermia in a volume of tissue (e.g., volume of tissue 260) which extends from the surface of skin (illustrated as skin depth 0 in FIG. 6). Sources corresponding to exemplary profiles 1-6 and 8-10 may also be used to achieve hyperthermia in a volume of tissue (e.g., volume of tissue 260) which extends from the surface of skin by suitably increasing the power of source to achieve a greater flux.

TABLE 2
Flux and minimum exposure time to heat body up to
+42° C. without active cooling.

| N | Wavelength, nm | Flux, W/cm² | Heating time, s |
|---|---|---|---|
| 1 | 800 | 0.683 | 209 |
| 2 | 925 | 0.573 | 193 |
| 3 | 960 | 0.466 | 206 |
| 4 | 1060 | 0.535 | 187 |
| 5 | 1208 | 0.383 | 189 |
| 6 | 1240 | 0.377 | 199 |
| 7 | 1440 | 0.491 | 208 |
| 8 | 1540 | 0.354 | 219 |
| 9 | 1730 | 0.359 | 212 |
| 10 | 2200 | 0.425 | 214 |

TABLE 3
Flux and minimum exposure time to heat skin up to
+42° C. with active cooling of skin
surface at the temperature +36° C.

| N | Wavelength, nm | Flux, W/cm² | Heating time, s |
|---|---|---|---|
| 1 | 800 | 1.76 | 41 |
| 2 | 925 | 1.135 | 36 |
| 3 | 960 | 1.085 | 47 |
| 4 | 1060 | 0.967 | 35 |
| 5 | 1208 | 0.643 | 37 |
| 6 | 1240 | 0.685 | 41 |
| 7 | 1440 | 3.39 | 170 |
| 8 | 1540 | 1.21 | 132 |
| 9 | 1730 | 0.996 | 124 |
| 10 | 2200 | 2.335 | 170 |

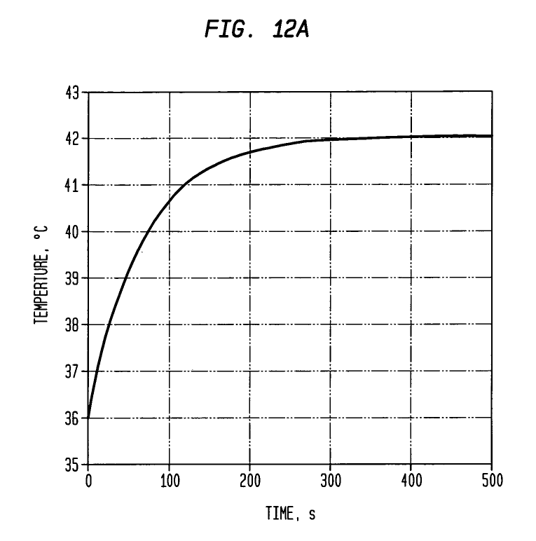

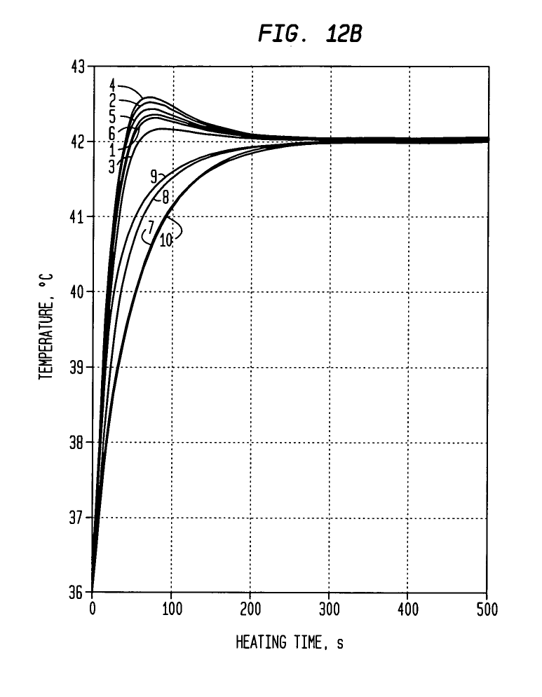

FIG. 12A illustrates the temperature at the skin surface as a function of time of exposure to a 800 nm radiation at a flux of 680 mW/cm², wherein the beam has a diameter larger than 2.5 cm. The data illustrated in FIG. 12A was calculated using a computer model including the following assumption: a 3 mm skin thickness, a 5 mm subcutaneous fat thickness, muscle extending below the subcutaneous fat, and a body temperature of 37° C. FIG. 12B illustrates temperature profiles corresponding to an embodiment of FIG. 2 in which the skin surface is cooled and kept to 36° C. The temperature profiles of FIG. 12B correspond to the data of Table 3. The data illustrated in FIG. 12B were calculated using a computer model including the following assumption: a 3 mm skin thickness, a 5 mm subcutaneous fat thickness, muscle extending below the subcutaneous fat, and a body temperature of 36° C.

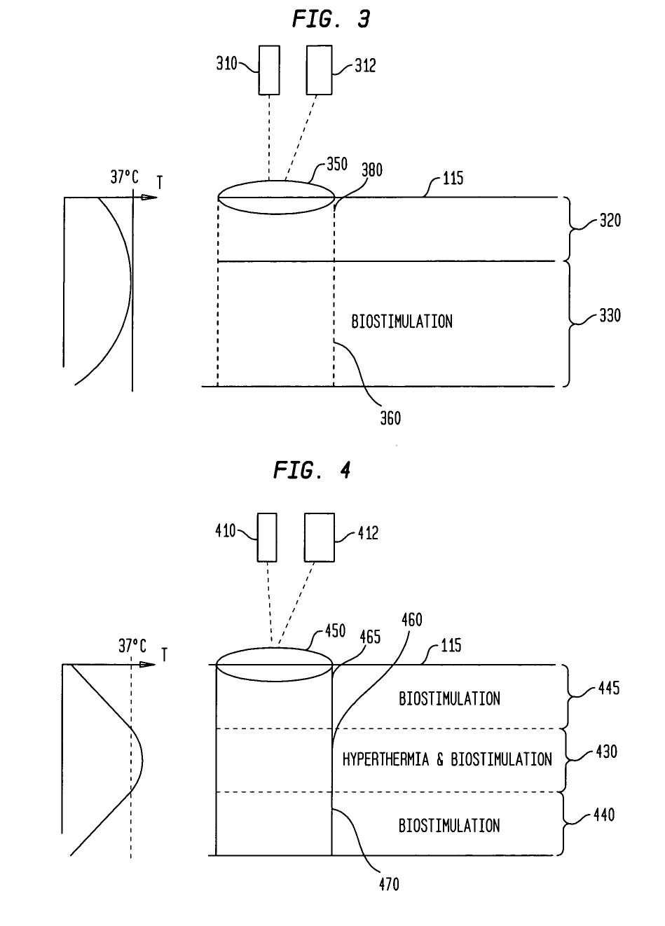

FIG. 3 illustrates a third aspect of the invention to generate photobiostimulation in a volume of tissue 360 in a depth region 330 below the surface of skin 115 and cooling is applied to the surface of skin 115. Photobiostimulation may be suppressed or reduced in efficacy in volume of tissue 380 in a depth region 320 by cooling surface of skin 115. Volume of tissue 360 is defined by depth region 330, and an area 350. Hyperthermia does not occur in any portion of volume of tissue 360.

To achieve photobiostimulation (without hyperthermia) in volume 360 with suppressed biostimulation or biostimulation of reduced efficacy in volume 380, a source 310 projects radiation in a 1-10,000 mW/cm² range and cooler 312 applies cooling at the skin surface to decrease temperature in a volume 380 defined by area 350 and depth region 320 to a hypothermic temperature (i.e., a temperature below normal body temperature). Cooler 312 can be any suitable cooler, for example a fan, flow of cold (below 36° C.) fluid (i.e., liquid or gas), cryogenic spray, vaporizing cream, cold plate or window in contact with skin, or other contact or non-contact cooler.

The temperature of the target region may be reduced to approximately 0-36° C., or about 10-36° C., or about 15-36° C., or about 20-36° C., or about 28-36° C. Hypothermia may be used to protect the skin from damage caused by heat generated by irradiation. Additionally, by reducing the temperatures, the efficacy of biostimulation may be reduced or biostimulation may be suppressed. A reduction in efficacy may be due to a variety of factors, including reduced mirocirculation of blood, and slowing down of relevant biochemical reactions with lower temperature. Cooling of the target region can slow down metabolic and physiological processes and reduce the oxygen need of cells, particularly neurons. Care must be taken to prevent frostbite, which can occur at temperatures below 0° C. In addition, the total body temperature (i.e., rectal temperature) should not be reduced below about 28° C., the point at which the ability to regain normal temperature is lost. In some embodiments, temperatures below 0° C. can be used on a small target area for short time periods.

In some aspects, hypothermia may result in increased biostimulation. Reducing temperature leads to the generation of specific cold shock proteins, phase transfer in lipid structure of cell membrane or fat cells. These changes to the target region can increase the efficacy of biostimulation for the treatment of specific diseases or cosmetic conditions.

For example, to achieve biostimulation without hyperthermia, irradiation with a wavelength of 500-1200 nm at a flux 1-1,000 mW/cm² and beam area of 0.8 cm² (e.g., a round area yielding a spot size at the target area of greater than 1 cm²), for a time interval greater than 60 seconds will achieve biostimulation to a depth of 25 mm. If the skin surface 115 is kept at 0-32° C., hypothermia will exist in a volume 380 above treatment region 360 resulting in reduced or suppressed biostimulation in this volume. In some embodiments, hypothermia can increase biostimulation.

FIG. 4 illustrates another aspect of the invention in which a volume of tissue 460 is heated such that biostimulation is applied to a hyperthermic volume of tissue 460, wherein volume of tissue 460 is at a selected depth below the surface of the skin 115, and volumes (without hyperthermia) 465, 470 are located above and below volume 460, respectively. Hyperthermia is suppressed in volume 465 by a cooler 412 and volume 470 is not heated sufficiently to achieve hyperthermia. Volume of tissue 460 is defined by depth region 430, and an area 450.

To achieve photobiostimulation and hyperthermia in volume 460, a source 410 projects radiation in a 100-10,000 mW/cm² range and cooler 412 applies cooling at the skin surface (0-30° C.) to suppress hyperthermia at surface 115. Treatments, such as the treatment of FIG. 4, may be achieved using a biostimulative source applied using a relatively large area of illumination (e.g., a round area having a spot size with a diameter larger than 1 cm-200 cm or more). Heating a volume of tissue wherein the volume is a selected depth below the surface of the skin is described in U.S. Provisional Application 60/389,871, filed Jun. 19, 2002, entitled “Method and Apparatus for Photothermal Treatment of Tissue at a Depth,” the substance of which is incorporated by reference herein.

For example, to achieve photobiostimulation and hyperthermia according to the present aspect of the invention, irradiation with a wavelength of 500-1250 nm at a flux 100-10,000 mW/cm² and a area of irradiation of 0.8 cm² after 60 seconds of exposure will achieve biostimulation in a range of depths 0-50 mm below the skin surface, and if the skin surface is kept at 0-30° C. hyperthermia will be achieved in a range of depths 0.2-30 mm below the skin surface. Treatments according to this aspect of the invention may be achieved using a relatively large area (e.g., a round area having a spot size diameter 1 cm-200 cm or more).

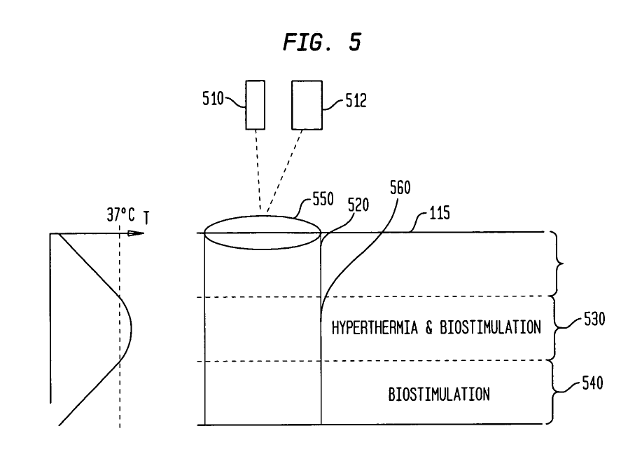

FIG. 5 illustrates another aspect of the invention in which a volume of tissue 560 is heated by source 510 such that enhanced biostimulation occurs in this hyperthermic volume of tissue, volume 560 being located a selected depth below the surface of the skin 115. The skin surface 550 can be cooled by the cooling source 512 either simultaneously or sequentially to the heating. Biostimulation (without hyperthermia) occurs in a volume 540 located below volume 560. A volume of tissue 560 is defined by depth region 530, and an area 550.

As described above with reference to FIG. 4, the efficacy of biostimulation is suppressed in a volume 520 adjacent to skin surface. However, according to this aspect of the invention, hyperthermia occurs only in volume 560.

For example, to achieve photobiostimulation and hyperthermia according this aspect of the invention, irradiation with a wavelength of 500-1250 nm at a flux 100-10,000 W/cm² and an area of irradiation greater than 0.8 cm² after 60 seconds of exposure will achieve biostimulation in a range of depths 0.1-50 mm below the skin surface, and if the skin surface is kept at 0-30° C., hyperthermia will be achieved in a range of depths 0.2-30 mm below the skin surface. Treatments according to this aspect of the invention may be achieved using a relatively large area (e.g., a round area having a spot size 1 cm-200 cm or more).

FIG. 7 depicts graphical data and corresponding tabular data, for achieving a selected temperature profile using exemplary wavelengths of monochromatic light, in which the skin surface is cooled to a temperature of 10° C. and photobiostimulation is suppressed in a region of tissue adjacent the skin surface. Specifically, the numbered entries in Table 3 describe the flux at the skin surface and the time necessary to achieve a correspondingly-numbered steady-state temperature profile in FIG. 7. It is to be understood that the wavelengths in FIGS. 6 and 7 are exemplary and light of any suitable wavelength may be used to achieve hyperthermia, and biostimulation.

Although the above discussion describes static (i.e., non-moving) radiation sources, the desired combination of photobiostimulation and photohyperthermia can be achieved by moving an output head of a radiation source across the surface of the skin so as to achieve the desired tissue temperature and/or deliver the desired amount of light to achieve biostimulation. The head may be moved over each skin surface area a single time or multiple times as required to achieve the desired therapeutic effect. Moving a source across the surface of the skin can be used to achieve hyperthermia in a volume of tissue due to the relatively long thermal relaxation time of bulk tissue. Further details regarding moving sources and heating of tissue is given in U.S. Pat. No. 6,273,884 B1 , entitled “Method and Apparatus for Dermatology Treatment,” to Altshuler et al., issued Aug. 14, 2001, the substance of which is hereby incorporated by reference. Photobiostimulation can be achieved by moving the source output head across the skin at a rate and/or for a number of iterations such that the desired number of photons are delivered to the treatment volume of tissue.

The above aspects of the invention are directed to applying biostimulation to a hyperthermic and/or a hypothermic volume of tissue. For these aspects, the heating source and biostimulative radiation source may be applied simultaneously, and for some embodiments may be the same source, or the heating source may be discontinued during application of the biostimulative radiation, or the heating source may be applied in a reduced amount to maintain the hyperthermic condition.

FIG. 8 is a schematic diagram of a light projection system 800 appropriate for use with aspects of the present invention according to FIG. 2 above. Light projection system 800 is composed of a radiation source 802 and a lens system 820. The radiation source may be any suitable narrowband source for generating hyperthermia and biostimulation according to an embodiment of the invention described above with reference to FIG. 2. For example, the source may be a laser (e.g., a continuous-wave diode laser, emitting at 805 nm with output power of 90 W) or an array of lasers, an LED (or an array of LEDs) or a lamp. The radiation from source 802 may be coupled to an optical fiber 803 (e.g., a 1 mm core quartz-polymer fiber) or a suitable fiber bundle, which is coupled on its proximal end to light source 802.

Lens system 820 may be any suitable lens system for transmitting light from source 802 to a patient's skin surface with a flux and beam size as described above with reference to FIG. 2. In one embodiment, lens system 820 includes a negative lens 806, and a positive lens 808 that forms a collimated output beam 810. In one embodiment of system 800, lens 806 is a refractive lens, and lens 808 is a Fresnel lens. A Fresnel lens may provide safety effects (e.g., a more uniform illumination pattern due to a reduction of speckle). As an example of this embodiment, lens 806 is a negative lens having a focal length of 25 mm and a diameter of 25 mm, and lens 808 is a 152 mm diameter Fresnel lens with a focal length 152 mm; and the distance between radiation source 802 and lens 806 is 20 mm, and the distance between the lenses 806 and 808 is 105 mm.

According to some aspects of the invention, output beams having larger diameters are used to direct narrowband light (e.g., laser or monochromatic filtered light) more deeply into the dermis and subcutaneous tissue than conventional low power laser sources emitting small beam sizes. For example, according to the above exemplary embodiment of lens system 820, for a 90 W source, lens system 820 produces an output beam 810 having a diameter of 160 mm, and has an output flux of 200 mW/cm² to 2000 mW/cm² (at a distance of 23 cm from lens 808).

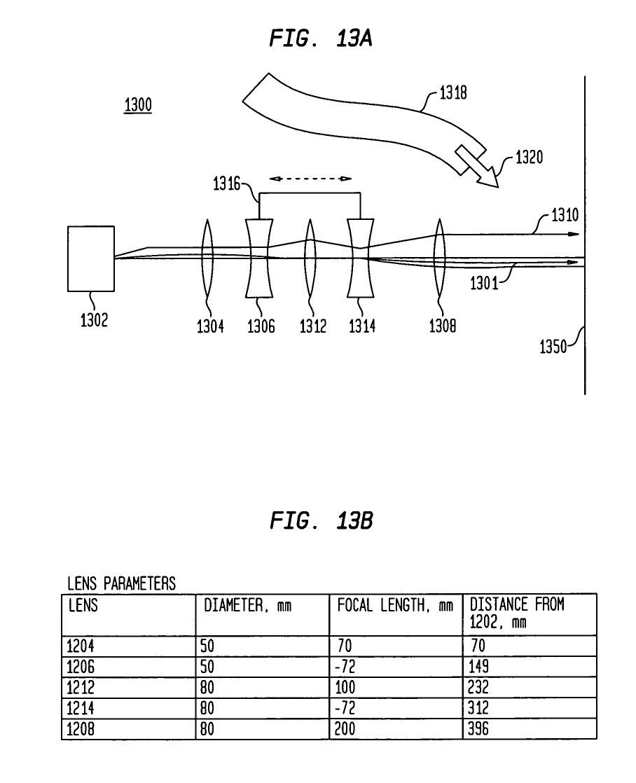

FIG. 13A is an exemplary embodiment of a light projection system 1300 according to aspects of the present invention, enabling one to practice the invention according to the scenarios illustrated in FIGS. 1 and 3, 4, and 5. For example, projection system 1300 may be any system that provides an output beam having suitable diameter and flux at skin surface 1350. In one embodiment, projection system 1300 includes an optical source 1302, and optical elements 1304, 1306, 1312, 1314, and 1308. One exemplary set of lens parameters is given in FIG. 13B.

Optical elements 1306 and 1314 may be movable along optical axis 1301 such that output beam 1310 has a variable diameter. For example, lenses 1306 and 1314 may be connected to a rigid frame 1316 (e.g., a translation stage), allowing synchronous movement of the lenses 1306 and 1314 along optical axis 1301 of the system 1300. Such movement provides variation in the beam width of the output beam 1310 (e.g., spot size is changed) and provides a corresponding variation in flux on skin surface 1350. For example, the system 1300 can provide continuous variations of a spot size between 4 cm and 8 cm, with the flux varying through a corresponding range of 7 W/cm² to 2 W/cm² (assuming source 1302 is a 90 W source). It is to be appreciated that by suitable selection of elements and source 1302, lens system 1300 may be designed to achieve any output beam 1310 as described herein, and any suitable output density as described herein.

System 1300 includes at least one air tube 1318, connected on its proximal ends to a cold or hot air source (not shown) and providing, at its distal end, an airflow 1320 directed at patient's skin 1350. For example, a total air flow from the at least one air tube 1318 may be at least 50 m³/min to vary air temperature in accordance with the embodiments illustrated in FIGS. 3-5 (e.g., the temperature will be between 0° C., and 45° C. at skin surface 1350); and in accordance with FIG. 1, a hot air flow will be provided to skin surface 1350. By varying the beam diameter and the air temperature, all regimens of FIGS. 1, 3, 4, and 5 can be realized using the system of FIG. 13A. While FIGS. 8 and 13A were described by specifying beam diameters, it is to be appreciated that by appropriate aperturing, any shape beam may be achieved.

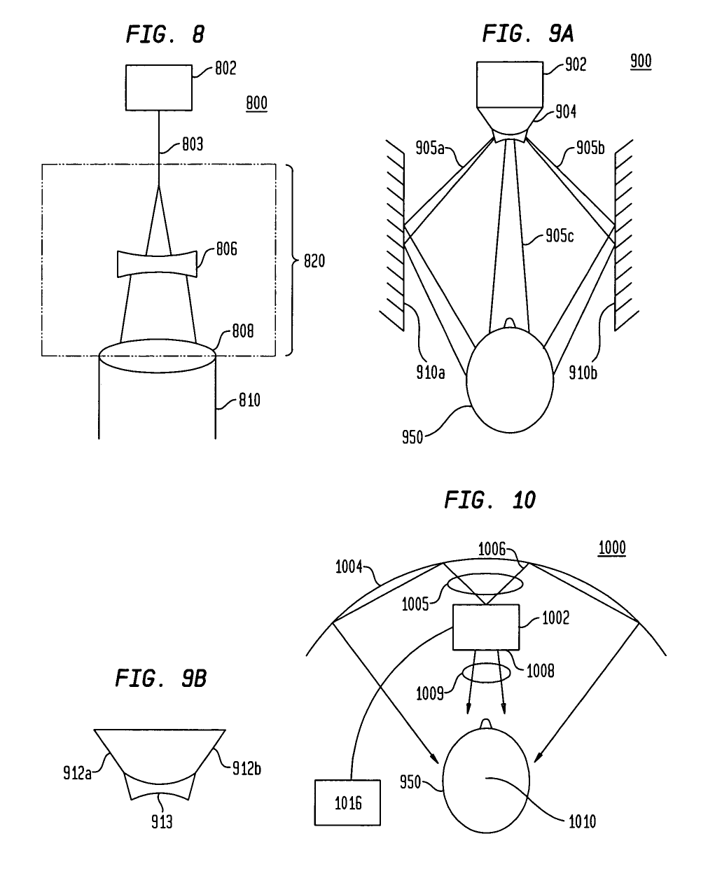

FIG. 9A is a first exemplary embodiment of a light projection system 900 for forming substantially uniform illumination over a non-flat surface 950, such as a patient's head or thigh. A collimated beam from a source 902 is directed onto a beam splitter 904 to form a plurality of beam portions 905a-c. In the illustrated embodiment, beam splitter 904 forms three component beam portions 905a 905b,and 905c however a light projection system 900 having two or more beam portions may provide advantages. Beam portion 905b is directed directly on the surface 950, and beam portions 905a and 905c are directed onto mirrors 910a and 910b, respectively, and then redirected to the sides of the surface 950. The clear apertures of beam splitter 904, mirrors 910a, 910b or additional apertures can be selected to achieve any desired area of irradiation on surface 950 (e.g., 1-200 cm²). Light projection system 900 may be modified (e.g., to treat one side of a patient's face) by blocking one of beams 910a and 910b.

FIG. 9B is a schematic of one example of a beam splitter 904. Beam splitter 904 is a prism having two flat surfaces 912a, 912b appropriately angled to direct light onto mirrors 910a, b, and a surface 913 having a negative power to expand light onto the front portion of surface 950.

FIG. 10 is a schematic of a second exemplary embodiment of a light projection system 1000 for forming substantially uniform illumination over a non-flat surface 950. Light projection system 1000 has a head 1002 adapted to project light in two directions. A first portion of light 1006 is directed in a first direction onto a curved reflector 1004 and then onto surface 950, and a second portion 1008 is directed in a second direction onto a surface 950. First portion of light 1006 is projected onto reflector 1004 directly or through an optical element (lens 1005), and second portion 1008 projected directly onto surface 950 or through an optical element (e.g., lens 1009).

Reflector 1004 may have any suitable shape for achieving a selected treatment. In some embodiments, reflector 1004 is designed such that center 1010 of surface 950 (e.g., the center of a patient's head) is located substantially at the center of curvature of reflector 1004. Alternatively, reflector 1004 may have an elliptical curvature and center 1010 of surface 950 (e.g., the center of a patient's head) is located substantially at a focus of reflector 1004 and the center 1010 of surface 950 is located at a second focus of reflector 1004. In one embodiment, reflector 1004 can be a diffuse reflector.

Projection system 1000 may include a control module 1016 comprising an electrical power source and control electronics. Additionally, a light source (not shown) may be mounted in head 1002; alternatively, a light source may be mounted in module 1016 and delivered to head 1002 by an optical fiber or a bundle of fibers. Light sources can be narrow band (e.g., diode lasers, LEDs), or broadband (e.g., filtered lamp). Alternatively, light sources may be a combination of narrow band and broadband sources. Optionally, in accordance with the embodiments described above, cold or hot air can be directed on the surface from head 1002 onto surface 950.

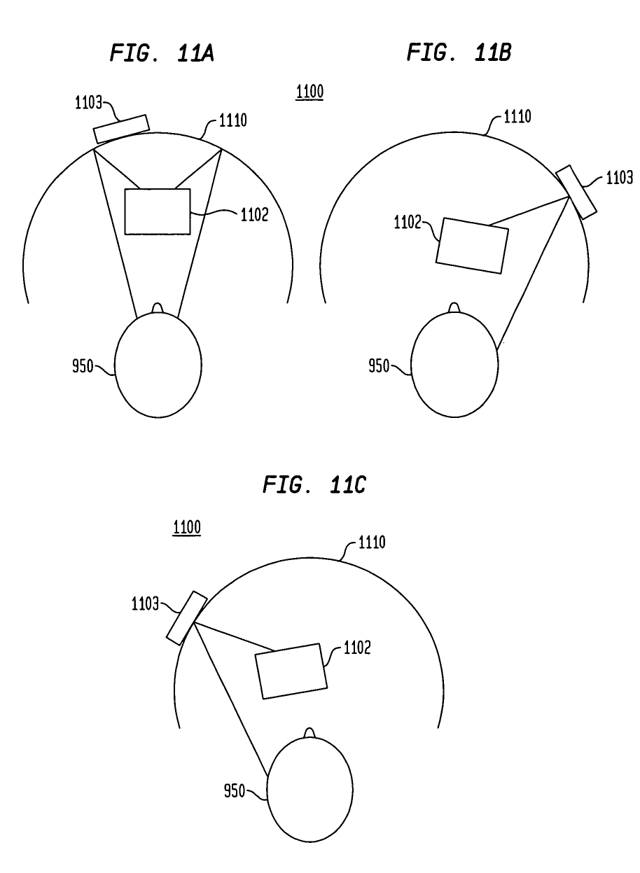

FIGS. 11A, 11B, and 11C are schematics of a third example of an embodiment of a light projection system 1100 for forming substantially uniform illumination over a non-flat surface 950 in which a rotatable head 1102 reflects light from a surface 1110 onto surface 950. In FIG. 11A, rotatable head 1102 is positioned such that light is directed onto the front portion of surface 950. In FIG. 11B, rotatable head 1102 is positioned such that light is directed onto a first side portion of surface 950. In FIG. 11C, rotatable head 1102 is positioned such that light is directed onto a second side portion of surface 950.

Optionally, head 1102 may be omitted, and replaced with a source mounted on surface 1110 such that the source is moved to various positions on surface 1110 to direct light onto each of the portions indicated in FIGS. 11A-11C. Alternatively, a plurality of sources can be mounted on surface 1110 and selectively illuminated to direct light onto each of the portions.

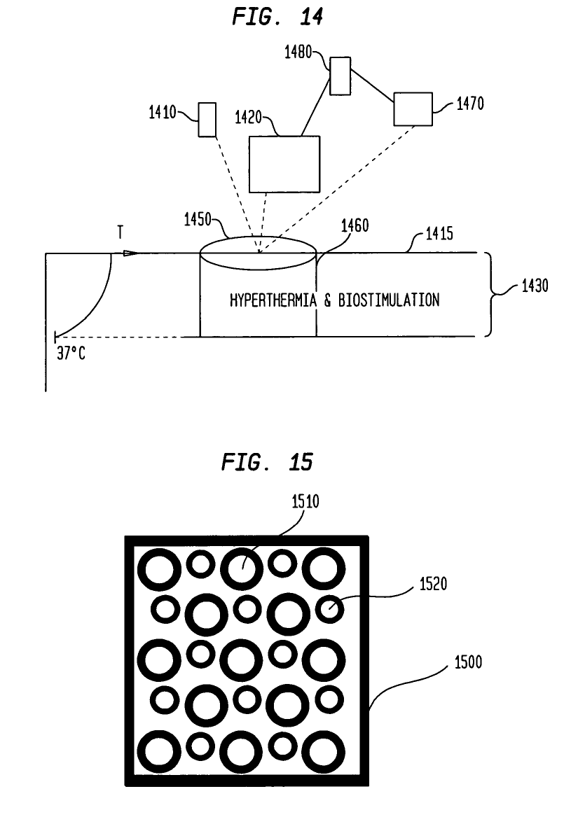

In another aspect, the present invention provides a feedback mechanism for controlling the temperature of a target region within a selected range while causing biostimulation within that target region and/or a volume above, below, or adjacent to the target region. The feedback mechanism can be used to control both heating and cooling of the target region. With reference to FIG. 14, in an exemplary embodiment, the source of electromagnetic radiation 1410 generates radiation for illuminating a portion of the surface area of the patient's skin 1450 so as to irradiate a volume of the patient's tissue 1460 that extends from the surface of the skin 1415 to a given depth 1430 below the skin. The radiation includes one or more wavelength components that can cause biostimulation of the irradiated tissue volume 1460. Another source 1420, for example, a separate source of electromagnetic radiation, controls the temperature of the irradiated volume, e.g., by illuminating the skin surface area 1450 with radiation having wavelength components suitable for heating tissue. A sensor 1470, for example, an optical pyrometer, measures the temperature of the illuminated skin portion 1450, and transmits the measured temperature to a feedback control circuitry 1480. The feedback circuitry 1480 compares the measured temperature with at least one threshold temperature, and transmits feedback signals, if needed, to the source 1420 based on this comparison. For example, if the measured temperature exceeds a pre-defined upper threshold, such as when the portion of the surface area of the patient's skin 1450 is heated to cause hyperthermia, the feedback circuitry can transmit a signal to the source 1420 to lower the amount of heat delivered to the skin portion 1450. Alternatively, the feedback circuitry can instruct the source 1420 to increase the amount of heat delivered to the skin portion 1450 if the measured temperature falls below a pre-defined lower threshold. In this manner, the temperature of the illuminated skin portion 1450, and consequently that of the target region 1460, can be actively maintained within a selected range about an operating temperature. For example, the above feedback mechanism can ensure that the operating temperature remains within ±1° C. of 39° C. A variety of sensors and feedback circuitry suitable for use in the practice of the invention are known in the art.

Those skilled in the art will appreciate, or be able to ascertain using no more than routine experimentation, further features and advantages of the invention based on the above-described embodiments. Accordingly, the invention is not to be limited by what has been particularly shown and described, except as indicated by the appended claims. The contents of all references, patents and published patent applications cited throughout this application, are incorporated herein by reference.

1. A method for biostimulating a target region of a subject, comprising:
irradiating the target region with a radiation, generated by a radiation source to have at least one selected wavelength component suitable for biostimulation, for a selected time duration, said time duration being chosen so as to cause biostimulation of said target region, and
controlling a temperature of said irradiated target region with a source independent of said biostimulating radiation so as to modulate efficacy of said biostimulation.

2. The method of claim 1, further comprising selecting said wavelength component to be in a range of about 380 nm to about 1250 nm.

3. The method of claim 1, further comprising selecting said wavelength component to be in a range of about 380 nm to about 600 nm.

4. The method of claim 1, further comprising selecting said wavelength component to be in a range of about 380 nm to about 450 nm.

5. The method of claim 1, further comprising selecting said wavelength component to be in range of about 600 nm to about 700 nm.

6. The method of claim 1, further comprising selecting said wavelength component to be in a range of about 760 nm to 880 nm.

7. The method of claim 1, wherein said radiation source generates radiation with a narrow bandwidth.

8. The method of claim 7, wherein said narrow bandwidth is less than about 100 nm.

9. The method of claim 1, further comprising selecting said time duration to be in a range of about 10 seconds to about one hour.

10. The method of claim 1, further comprising selecting said time duration to be in a range of about 10 minutes to about one hour.

11. The method of claim 1, wherein said radiation delivers a power flux in a range of about 1 to about 250 mW/cm² to said target region.

12. The method of claim 1, wherein said radiation delivers a power flux in a range of about 10 to about 100 mW/cm² to said target region.

13. The method of claim 1, wherein said radiation delivers an energy flux in a range of about 1 Joule/cm² to about 1000 Joules/cm² to said irradiated target region during said time duration.

14. The method of claim 1, wherein said radiation delivers an energy flux in a range of about 1 Joule/cm² to about 100 Joules/cm² to said irradiated target region during said time duration.

15. The method of claim 1, wherein irradiating said target region comprises exposing said target region to a beam of radiation having a cross-sectional area in a range of about 1 cm² to about 200 cm².

16. The method of claim 1, wherein the step of controlling temperature comprises heating said irradiated target region so as to increase efficacy of said biostimulation.

17. The method of claim 16, wherein said heating step is selected from the group consisting of contact heating, convection, and application of electromagnetic radiation.

18. The method of claim 16, wherein said heating step comprises applying ultrasound to said irradiated target region.

19. The method of claim 16, wherein said heating step comprises applying microwave radiation to said irradiated target region.

20. The method of claim 16, wherein said heating raises the temperature of said target region to a value in a range of about 37° C. to about 50° C.

21. The method of claim 16, wherein said heating raises the temperature of said target region to a value in a range of about 37° C. to about 45° C.

22. The method of claim 16, wherein said heating raises the temperature of said target region to a value in a range of about 37° C. to about 42° C.

23. The method of claim 1, wherein the step of controlling temperature comprises cooling said target region to decrease efficacy of said biostimulation.

24. The method of claim 23, wherein said cooling lowers the temperature of said target region to a value in a range of abut 0° C. to about 36° C.

25. The method of claim 23, wherein said cooling lowers the temperature of said target region to a value in a range of about 15° C. to about 36° C.

26. The method of claim 1, wherein controlling the temperature comprises utilizing a separate radiation source to heat said target region irradiated with biostimulating radiation.

27. The method of claim 26, wherein said separate radiation source comprises a narrowband source.

28. The method of claim 26, wherein said separate radiation source comprises a broadband source.

29. The method of claim 26, wherein said separate radiation source generates radiation having one or more wavelength components in a range of about 380 nm to about 2700 nm.

30. The method of claim 26, wherein said separate radiation source generates radiation having one or more wavelength components in a range of about 1000 nm to about 1250 nm.

31. The method of claim 26, wherein said separate radiation source generates radiation having one or more wavelength components in a range of about 700 nm to about 900 nm.

32. The method of claim 1, wherein controlling the temperature comprises placing said target region in thermal contact with a surface having a selected temperature.

33. The method of claim 1, wherein controlling the temperature comprises generating a flow of a fluid over said target region to be in thermal contact therewith.

34. The method of claim 1, wherein controlling the temperature comprises applying a vaporizing cream to said target region.

35. The method of claim 1, wherein said target region is disposed at a depth below a skin surface of the subject.

36. The method of claim 1, wherein the step of controlling the temperature of said irradiated target region comprises heating a first selected portion of the target region and cooling a second selected portion of the target region.

37. The method of claim 36, wherein the heating and cooling steps are simultaneous.

38. The method of claim 36, wherein the heating and cooling step are sequential.

39. A method of biostimulating a target region of a patient disposed at a depth below the patient's skin, comprising:
exposing a portion of the patient's skin for a selected time duration to a radiation having at least one wavelength component capable of penetrating to a depth associated with said target region so as to irradiate said target region, said wavelength component and said time duration being chosen to cause biostimulation within said target region, and
controlling a temperature of a volume of said patient through at least a portion of which said radiation traverses to reach said target region so as to modulate biostimulation within said volume relative to said target region.

40. The method of claim 39, wherein controlling the temperature comprises cooling said volume to decrease biostimulation therein.

41. The method of claim 39, wherein said cooling lowers a temperature of said volume to a value in a range of about 0° C. to about 36° C.

42. The method of claim 39, wherein said cooling lowers a temperature of said volume to a value in a range of about 15° C. to about 36° C.

43. The method of claim 39, wherein cooling said volume comprises cooling a portion of the patient's skin in proximity of said volume.

44. The method of claim 39, further comprising selecting said wavelength component to be in a range of about 380 nm to about 1250 nm.

45. The method of claim 39, further comprising selecting said wavelength component to be in a range of about 380 nm to about 600 nm.

46. The method of claim 39, further comprising selecting said wavelength component to be in a range of about 380 nm to about 450 nm.

47. The method of claim 39, further comprising selecting said wavelength component to be in a range of about 600 nm to about 700 nm.

48. A device for biostimulating a patient's target region, comprising:
a first source for generating electromagnetic radiation having one or more wavelength components suitable for causing biostimulation in said target region,
a radiation guidance device optically coupled to said source for delivering said radiation to the target region, and
a second source in communication with said target region for controlling a temperature of said target region in order to modulate efficacy of biostimulation caused by said electromagnetic radiation.

49. The device of claim 48, wherein said first source generates radiation having a bandwidth less than about 100 nm.

50. The device of claim 48, wherein said first source generates a substantially monochromatic radiation.

51. The device of claim 48, wherein said first source generates radiation having one or more wavelength components in a range of about 380 nm to about 1250 nm.

52. The device of claim 48, wherein said second source comprises a source of electromagnetic radiation generating radiation suitable for heating said target region so as to enhance the efficacy of biostimulation.

53. The device of claim 52, wherein said second source generates radiation having one or more wavelength components in a range of about 380 nm to about 2700 nm.

54. The device of claim 52, wherein the radiation guidance device comprises a lens system for delivering the biostimulating radiation from the first source to the target region.

55. The device of claim 52, wherein said lens system comprises a Fresnel lens.

56. The device of claim 52, further comprising an optical fiber coupled at an input thereof to said first radiation source and an output thereof to said lens system so as to direct light generated by said radiation source to said lens system.

57. The device of claim 52, wherein said lens system comprises at least one movable lens to allow adjusting a cross-sectional area of a radiation beam generated by said first source for irradiating said target region.

58. The device of claim 48, wherein said radiation guidance device comprises
a beam splitter adapted to receive a radiation beam from said first source in order to generate a plurality of beam portions, and
one or more reflective surfaces optically coupled to said beam splitter to direct one or more of said beam portions to a surface of the patient's skin so as to irradiate said target region.

59. The device of claim 58, wherein said reflective surfaces allow a substantially uniform illumination of said skin surface.

60. The device of claim 58, wherein said beam splitter comprises a prism.

61. The device of claim 58, wherein at least one of said reflective surfaces exhibits a curved profile.

62. A method of biostimulating a subject's target region, comprising
irradiating the target region with radiation having one or more wavelength components suitable for causing biostimulation within said target region, and
actively controlling a temperature of at least a portion of said target region to ensure it remains within a pre-defined range of an operating temperature in order to modulate efficacy of biostimulation with said target region.

63. The method of claim 62, wherein the step of actively controlling the temperature comprises measuring a temperature of a portion of the patient's skin in thermal contact with said target region.

64. The method of claim 63, wherein the step of actively controlling the temperature comprises comparing said measured temperature with at least one pre-defined threshold.

65. The method of claim 64, wherein the step of actively controlling the temperature comprises modifying an amount of heat delivered to or extracted from said target region in response to said comparison of the measured temperature with the pre-defined threshold.

66. A method for biostimulating a plurality of target regions of a subject, comprising
moving a radiation source over a portion of the subject's skin so as to irradiate sequentially a plurality of target regions with radiation having at least one wavelength component suitable for causing biostimulation, said moving of radiation source being performed at a rate selected to expose each of said regions to sufficient radiation for causing biostimulation within that region, and
controlling temperature of said target regions by a source independent of said biostimulating radiation so as to modulate efficacy of biostimulation within each of said target regions.

67. The method of claim 66, further comprising moving the radiation source continuously over said skin portion.

68. The method of claim 66, further comprising moving the radiation source at least twice over said skin portion.
